(19) United States
(12) Patent Application Publication
(10) Pub. No.: US 2019/0094417 A1
(43) Pub. Date: Mar. 28, 2019

(54) OPHTHALMIC DEVICES INCLUDING POLYDOPAMINE LAYERS AND METHODS OF DEPOSITING A METAL LAYER ON OPHTHALMIC DEVICES INCLUDING A POLYDOPAMINE LAYER

(71) Applicant: Verily Life Sciences LLC, Mountain View, CA (US)

(72) Inventors: Scott B. Kennedy, Mountain View, CA (US); Christian Gutierrez, San Francisco, CA (US)

(21) Appl. No.: 16/139,300

(22) Filed: Sep. 24, 2018

Related U.S. Application Data
(60) Provisional application No. 62/563,520, filed on Sep. 26, 2017.

(51) Int. Cl. G02B 1/04; G02B 1/10; A61L 27/30; A61L 27/34

(52) U.S. Cl. CPC G02B 1/043; G02B 1/10; A61L 27/306; A61L 2420/08; A61L 2430/16; A61L 2420/02; A61L 27/34

(57) ABSTRACT

A method for forming metal layers onto ocular lenses using polydopamine layers is described herein. Ophthalmic devices including a polydopamine layer and a metal layer disposed thereon are also described herein.

### CROSS-REFERENCE(S) TO RELATED APPLICATIONS

This application claims the benefit of U.S. Provisional Application No. 62/563,520, filed Sep. 26, 2017, which is hereby incorporated by reference in its entirety.

### TECHNICAL FIELD

This disclosure relates generally to ophthalmic devices and in particular but not exclusively, relates to methods of forming a metal layer on ophthalmic devices.

### BACKGROUND INFORMATION

Smart contact lenses frequently include a microelectronic assembly that is fitted, formed, and encapsulated within geometric constraints of an ocular lens. Such ocular lenses may include a generally curved surface, such as a generally curved surface configured to conformably contact portion of an eye. The microelectronic assembly frequently takes the form of a ring due to the design of a circular antenna and an open center so as to not occlude vision through the central aperture of the microelectronic assembly. Currently such a ring-shaped microelectronic assembly is fabricated on a flat substrate that serves as a carrier during the execution of a series of surface-based micro-fabrication processes. This results in the fabrication of a flat microelectronic assembly that requires additional steps to form or otherwise couple the microelectronic assembly in a curved state within the volume of contact lens. This forming process can introduce unwanted buckling resulting in electronic and mechanical failures. Furthermore, the need to form the electronics necessitates the use of thin thermoplastic substrate materials, which imposes thermal and mechanical constraints. Additionally, significant stress is experienced by non-compliant rigid areas of the assembly, such as bonded chips, sensors, and batteries. These components are attached in a flat configuration and are not amenable to downstream conformal fixation to a curved surface. Thus current methods of coupling microelectronic assemblies to a contact lens substrate result in a higher-stress, lower-reliability configurations, which are problematic for achieving, for example, a reliable, high-volume lens manufacturing process.

### BRIEF DESCRIPTION OF THE DRAWINGS

Non-limiting and non-exhaustive embodiments of the invention are described with reference to the following figures, wherein like reference numerals refer to like parts throughout the various views unless otherwise specified. Not all instances of an element are necessarily labeled so as not to clutter the drawings where appropriate. The drawings are not necessarily to scale, emphasis instead being placed upon illustrating the principles being described.

FIG. 1A is a top-down plan view of an ophthalmic device, in accordance with an embodiment of the disclosure.

FIG. 1B is a cross-sectional view of the ophthalmic device illustrated in FIG. 1A, in accordance with an embodiment of the disclosure.

FIG. 2 schematically illustrates a method of forming a metal layer on a surface of an ocular lens, in accordance with an embodiment of the disclosure.

FIG. 3 illustrates in cross-section an ophthalmic device including a metal layer on an anterior-facing surface and a metal layer on a posterior-facing surface of an ocular lens, in accordance with an embodiment of the disclosure.

FIG. 4 illustrates in cross-section another ophthalmic device including a metal layer disposed on a locally flat mesa on an anterior-facing surface and a metal layer disposed on a locally flat mesa on a posterior-facing surface of an ocular lens, in accordance with an embodiment of the disclosure.

FIG. 5 illustrates in cross-section another ophthalmic device including a metal layer disposed on a locally flat recess on an anterior-facing surface and a metal layer disposed on a locally flat recess on a posterior-facing surface of an ocular lens, in accordance with an embodiment of the disclosure.

FIG. 6 illustrates in cross-section an ophthalmic device including an electrically conductive via, in accordance with an embodiment of the disclosure.

FIGS. 7A-7C schematically illustrate a method of forming a metal layer on a surface of an ocular lens, in accordance with an embodiment of the disclosure.

FIGS. 8A-8C schematically illustrate a method of forming a metal layer on a surface of an ocular lens, in accordance with an embodiment of the disclosure.

FIGS. 9A-9C schematically illustrate a method of forming a metal layer on a surface of an ocular lens, in accordance with an embodiment of the disclosure.

FIGS. 10A-10E are images of ophthalmic devices at various stages of manufacturing by methods in accordance with embodiments of the disclosure.

FIG. 11 is a perspective view of an ophthalmic device, in accordance with an embodiment of the disclosure.

### DETAILED DESCRIPTION

Embodiments of a system, apparatus, and method of forming a metal layer disposed on a polydopamine layer on a surface of an ophthalmic device are described herein. For example, the method may include forming a polydopamine layer on one or more surfaces of an ocular lens and contacting the polydopamine layer with a metal precursor solution to form the metal layer. Further, the ophthalmic device may include an ocular lens; a polydopamine layer disposed on at least a portion of one or more surfaces of the ocular lens; and a metal layer disposed on the polydopamine layer.

In the following description numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Many ophthalmic devices include metal layers on surfaces of the ophthalmic device, including curved surfaces. Metal layers for ophthalmic devices are frequently formed on flat surfaces and then applied to a curved surface of the ophthalmic device. As discussed further herein, stress is induced in metal layers that have been formed on flat surfaces when they are placed on curved surfaces, such as those present on ophthalmic devices. As discussed further herein with respect to the methods of the present disclosure, metal layers can be deposited on curved surfaces of ophthalmic devices with a polydopamine layer disposed thereon. Such deposition results in lower stress induced in the metal layers. Accordingly, in an aspect, the present disclosure provides ophthalmic devices shaped for mounting in or on an eye comprising: an ocular lens; a polydopamine layer on a portion of one or more surfaces of the ocular lens; and a metal layer disposed on the polydopamine layer.

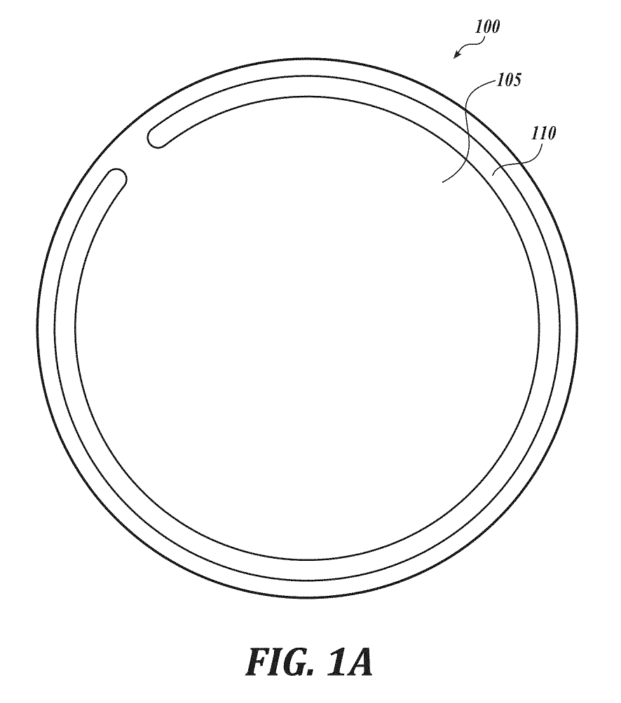

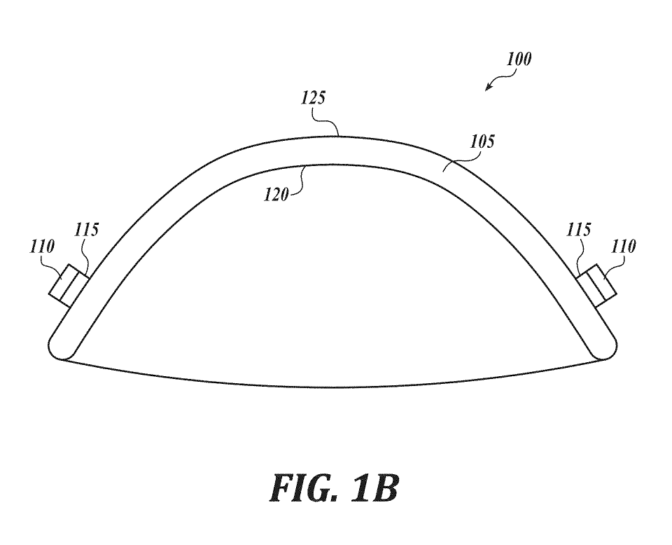

Turning to FIGS. 1A and 1B, an ophthalmic device 100 in accordance with an embodiment of the present disclosure is shown. FIG. 1A is a top-down plan view of ophthalmic device 100. FIG. 1B is a cross-sectional view of the ophthalmic device 100. In the illustrated embodiment, ophthalmic device 100 includes ocular lens 105 including lens material, such as a rigid core, and a metal layer 110 disposed around a periphery of ocular lens 105. In one embodiment, the ocular lens 105 includes a rigid material, such as polymethylmethacrylate. In another embodiment, the ocular lens 105 includes a soft or gas-permeable material. The soft or gas-permeable material may be fabricated from a hydrogel, such as a silicone hydrogel. As shown in FIG. 1B, ocular lens 105 includes an anterior-facing surface 125, a posterior-facing surface 120, a polydopamine layer 115 disposed on anterior-facing surface 125 of the ocular lens 105, and a metal layer 110 carried by the polydopamine layer 115. As used herein, a “polydopamine layer” refers to a layer of material including polydopamine. In an embodiment, the metal layer 110 is disposed around a periphery of the ocular lens 105, for example, to provide an optically transmissive center of the ocular lens 105. In that regard, a user can see through the ophthalmic device 100 when in use, such as when mounted to an eye.

Ophthalmic device 100 is shaped to be mounted on or in an eye. In an embodiment, ocular lens 105 of ophthalmic device 100 is shaped to be mounted to a surface of an eye, such as a corneal surface, as a contact lens. In an embodiment, ocular lens 105 is shaped to be implanted or otherwise disposed in an eye, such as in a capsular bag of an eye, as an intraocular lens.

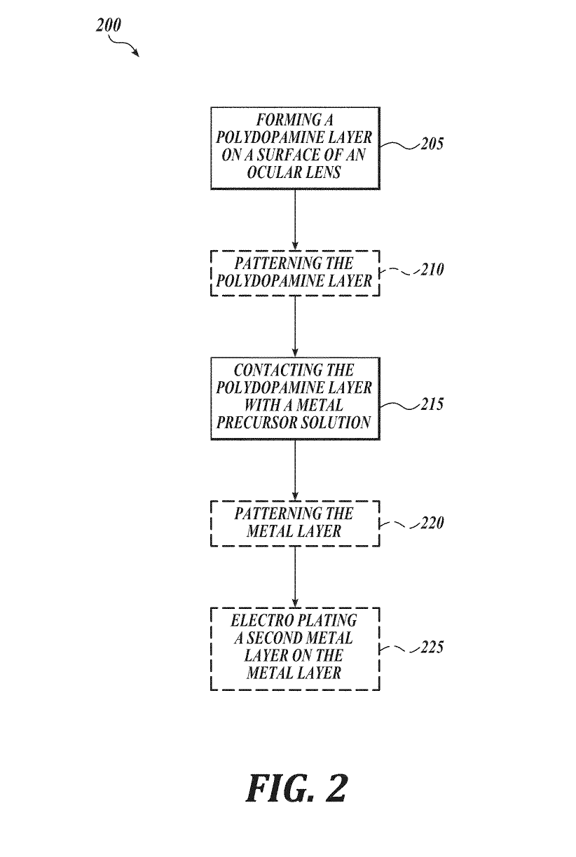

In an embodiment, the metal layer 110 is formed according the methods of the present disclosure discussed further herein with respect to FIGS. 2, 7-9, and 10A-10E, wherein the metal layer 110 is formed on polydopamine layer 115. As discussed further herein, this is in contrast to conventional methods in which, for example, a metal layer is formed on a flat surface not on the ophthalmic device 100 and later transferred to a curved surface of the ocular lens 105. As shown, the anterior-facing surface 125 and the posterior-facing surface 120 of the ocular lens 105 are generally curved. By forming the metal layer 110 in situ on polydopamine layer 115, stresses in metal layer 110 are reduced compared to if metal layer 110 were formed on a flat surface and placed on the generally curved surfaces of ocular lens 105.

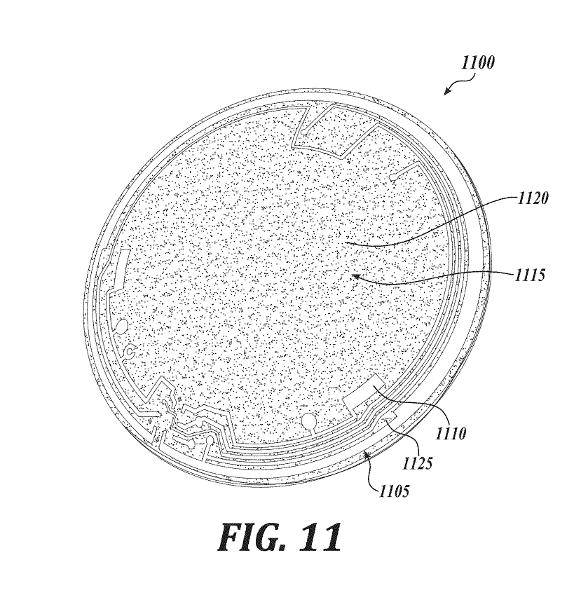

The ophthalmic devices of the present disclosure can include smart contact lenses, accommodating contact lenses, accommodating intraocular lenses, and the like. In an embodiment, the ophthalmic device of the present disclosure is an accommodating ophthalmic device. In that regard, attention is directed to FIG. 11 in which an accommodating ophthalmic device 1100 in accordance with an embodiment of the disclosure is illustrated. As shown, ophthalmic device 110 includes a metal layer 1125 in the form of a microelectronic assembly. Metal layer 1125 is carried by a polydopamine layer (not shown, see for example FIGS. 1A and 1B). In the illustrated embodiment the metal layer 1125 is disposed adjacent to a periphery 1105 of the ophthalmic device. Ophthalmic device 1100 further includes an accommodation actuator 1120 disposed in a center region 1115 of ophthalmic device configured to provide optical accommodation. Ophthalmic device 1100 further includes controller 1110 in conductive communication with accommodation actuator 1120 through the metal layer 1125. Controller 1100 includes logic that when executed by the controller 1110 causes the ophthalmic device 1100 to modulate the optical power of the ophthalmic device with the accommodation actuator 1120.

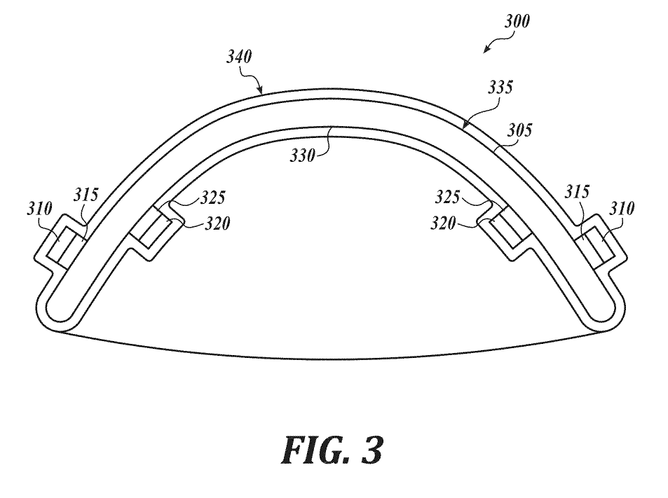

In an embodiment, the ophthalmic devices of the present disclosure include a polydopamine layer disposed on an anterior surface and a posterior surface of the ophthalmic device. In that regard, attention is directed to FIG. 3, in which an ophthalmic device in accordance with an embodiment of the disclosure is illustrated. FIG. 3 illustrates in cross-section an ophthalmic device 300 including a metal layer 310 on an anterior-facing surface 305 and a metal layer 320 on a posterior-facing surface 330 of the ocular lens 335, in accordance with an embodiment of the disclosure. While anterior-facing and posterior-facing are used herein to discuss the opposite sides of the ocular lens, for example, the anterior-facing and posterior-facing designations do not necessarily denote any directionality to the ocular lens and are used merely as a reference with respect FIGS. 1 and 3-6. In an embodiment, the anterior-facing surface is configured to oppose an eye when the ocular lens is in use, such as when mounted onto an eye. In an embodiment, the posterior-facing surface is configured to face an eye when the ocular lens is in use, such as when mounted onto an eye.

As shown, ophthalmic device 300 includes a posterior-facing surface 330, an anterior-facing surface 305, a polydopamine layer 315 disposed on the anterior-facing surface 305, a polydopamine layer 325 disposed on the posterior-facing surface 330; and metal layers 310 and 320 disposed on polydopamine layers 315 and 325, respectively. In an embodiment, polydopamine layers 315 and 325 are made according to method 200, discussed elsewhere herein, such as by process block 205. In the illustrated embodiment, ocular lens 335, polydopamine layers 315 and 325, and metal layers 310 and 320 are encapsulated in a hydrogel layer 340 amenable to contact an eye of a user without damaging the eye and providing physical separation between the eye and, for example, metal layers 310 and 320.

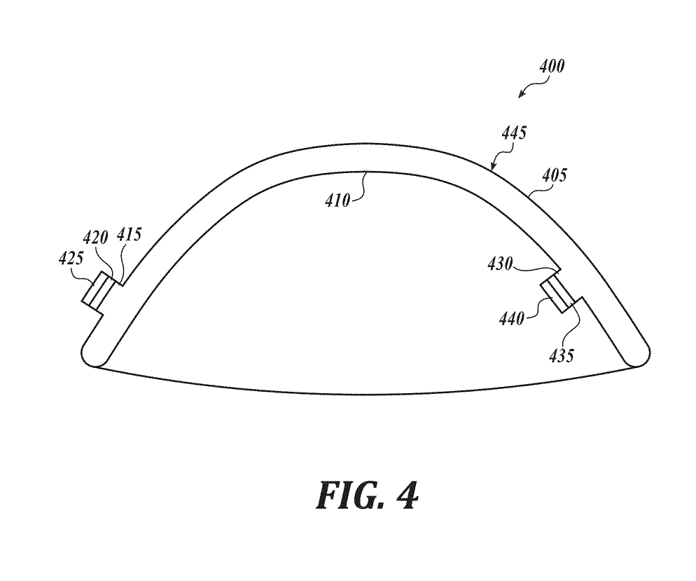

In an embodiment, the ophthalmic devices of the present disclosure include generally curved anterior-facing and posterior-facing surfaces, one or more locally flat portions of the anterior-facing surface and/or posterior-facing surface of the ophthalmic device, wherein at least a portion of the polydopamine layer is disposed on the one or more locally flat portions. In that regard, attention is directed to FIG. 4, which illustrates in cross-section another ophthalmic device 400, in accordance with an embodiment of the disclosure. Ocular lens 445 includes generally curved posterior-facing surface 410, polydopamine layer 435 disposed on the posterior-facing surface 410, metal layer 440 disposed on polydopamine layer 435, generally curved anterior-facing surface 405, polydopamine layer 420 disposed on the anterior-facing surface 405, and metal layer 425 disposed on the polydopamine layer 420.

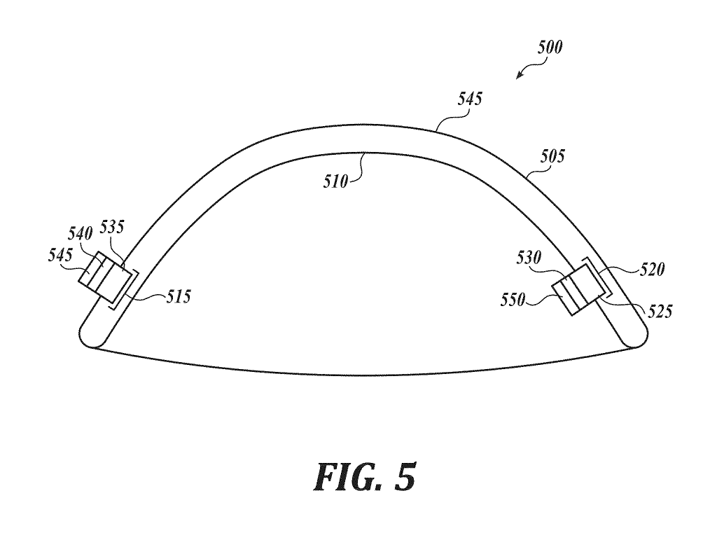

In the illustrated embodiment, ocular lens 400 includes a locally flat portion 430 disposed on the posterior-facing surface 410 the ocular lens 400. As shown, the locally flat portion 430 extends above the generally curved posterior-facing surface 410 to define a mesa. Likewise, anterior-facing surface 405 includes a locally flat portion 415, which extends above the generally curved anterior-facing surface 405 to define a mesa. As such, only a portion of the surface of ophthalmic device 400 is flat, whereas other portions of ophthalmic device 400 including anterior-facing surface 405 and posterior-facing surface 410 are generally curved. As shown, each mesa 430 and 415 carry a polydopamine layer 435 and 420, respectively, which in turn carry a metal layer 440 and 425. In this regard, the metal layers 425 and 440 are configured to carry an electronic component including a generally flat surface coupled thereto. As discussed further herein with respect to FIG. 5, such coupling of electronic components including generally flat surfaces generally avoids or mitigates stresses associated with, for example, coupling such flat electronic components to a curved surface of the ocular lens 400.

In an embodiment, the ophthalmic devices of the present disclosure include one or more flat portions that are recessed from a generally curved anterior-facing and/or a generally curved posterior-facing surface of the ocular lens. In that regard, attention is directed to FIG. 5, which illustrates in cross-section another ophthalmic device 500, in accordance with an embodiment of the disclosure. Ophthalmic device 500 includes ocular lens 545 and including generally curved posterior-facing surface 510, polydopamine layer 525 disposed on the posterior-facing surface 510, metal layer 530 disposed on the polydopamine layer 525, generally curved anterior-facing surface 505, polydopamine layer 535 disposed on the anterior-facing surface 505, and metal layer 540 disposed on the polydopamine layer 535. In the illustrated embodiment, ophthalmic device 500 includes a locally flat portion 520 disposed on the posterior-facing surface 510 the ocular lens 545. As shown, the locally flat portion 520 is recessed from the generally curved posterior-facing surface 510 to define a locally flat portion 520 on to which polydopamine layer 525 is disposed Likewise, locally flat portion 515 is recessed from generally curved anterior-facing surface 505 to define a locally flat recess 515 on which polydopamine layer 535 is disposed. Further, as shown, the locally flat portions 515 and 520 do not extend over the entire surfaces of ophthalmic device 500, but are instead flat over only a portion of the surfaces of ophthalmic device 500.

Like the locally flat mesas, the locally flat recesses 520 and 515 are configured to carry polydopamine layers 525 and 515 and metal layers 530 and 540, respectively. In this regard, the metal layers 540 and 530 are configured to carry electronic components having generally flat surfaces without inducing stresses associated with, for example, coupling the generally flat surfaces to a curved surface of the ocular lens 500. In the illustrated embodiment, ophthalmic device 400 includes electronic components 550 and 545 including generally flat surfaces coupled to a portion of the metal layers 540 and 530 disposed on the polydopamine layers 535 and 525 disposed on the locally flat portions 515 and 520, respectively.

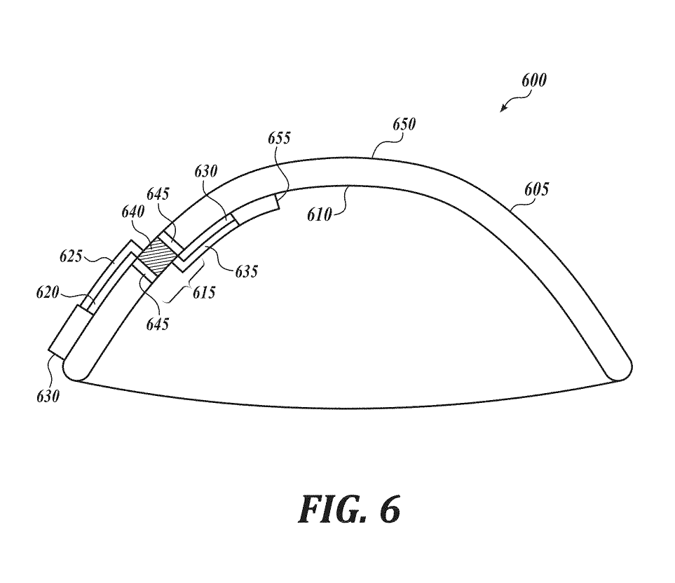

In an embodiment, the ophthalmic devices of the present disclosure include an ocular lens including an electrically conductive via. In that regard, attention is directed to FIG. 6, which illustrates an ophthalmic device 600 including an aperture 615, in accordance with an embodiment of the disclosure. In the illustrated embodiment, ophthalmic device 600 includes ocular lens 650 including aperture 615 extending through ocular lens 650 between posterior-facing surface 610 and anterior-facing surface 605, a polydopamine layer 630 disposed on posterior-facing surface 610, a polydopamine layer 620 disposed on anterior-facing surface 605, and an aperture polydopamine layer 645 disposed on a surface of ocular lens 650 that defines aperture 615. As shown, ophthalmic device 600 includes aperture metal layer 640 disposed on aperture polydopamine layer 645 and, in certain embodiments, filing the aperture 615 to provide an electrically conductive via between anterior-facing surface 605 and posterior-facing surface 610. In an embodiment and as illustrated, metal layer 625 carried by polydopamine layer 620 on anterior-facing surface 605 and metal layer 635 carried by polydopamine layer 630 on the posterior-facing surface 610 are in conductive communication with aperture metal layer 640.

As shown, ophthalmic device 600 includes a first electronic component 655 disposed on the posterior-facing surface 610 and a second electronic component 660 disposed on the anterior-facing surface 605 in conductive communication through the conductive aperture 615. In this regard, a sensor, such as first electronic component 665, positioned to contact an eye of a user when ophthalmic device 600 is, for example, mounted on an eye can be in conductive communication with a transceiver, such as second electronic component 660, configured to transmit data generated by the sensor 665.

In another aspect, the present disclosure provides a method of forming a metal layer on a surface of an ocular lens, such as an ocular lens of an ophthalmic device. In an embodiment, the method comprises forming a polydopamine layer on one or more surfaces of the ocular lens; and contacting the polydopamine layer with a metal precursor solution to form the metal layer. As described further herein, by forming metal layers on polydopamine layers deposited on surfaces of an ocular lens, stresses on the metal layers may be reduced, particularly when the surfaces of the ocular lens are curved or otherwise non-planar.

FIG. 2 illustrates an example method 200 of forming a metal layer on one or more surfaces of an ocular lens, in accordance with an embodiment of the disclosure. The method may be used to form the metal layers illustrated in FIGS. 1, 3-6, and 11. The order in which some or all of the process blocks appear in method 200 should not be deemed limiting. Rather, one of ordinary skill in the art having the benefit of the present disclosure will understand that some of the process blocks may be executed in a variety of orders not illustrated, or even in parallel.

The method 200 may begin with process block 205, which includes forming a polydopamine layer on a surface of an ocular lens.

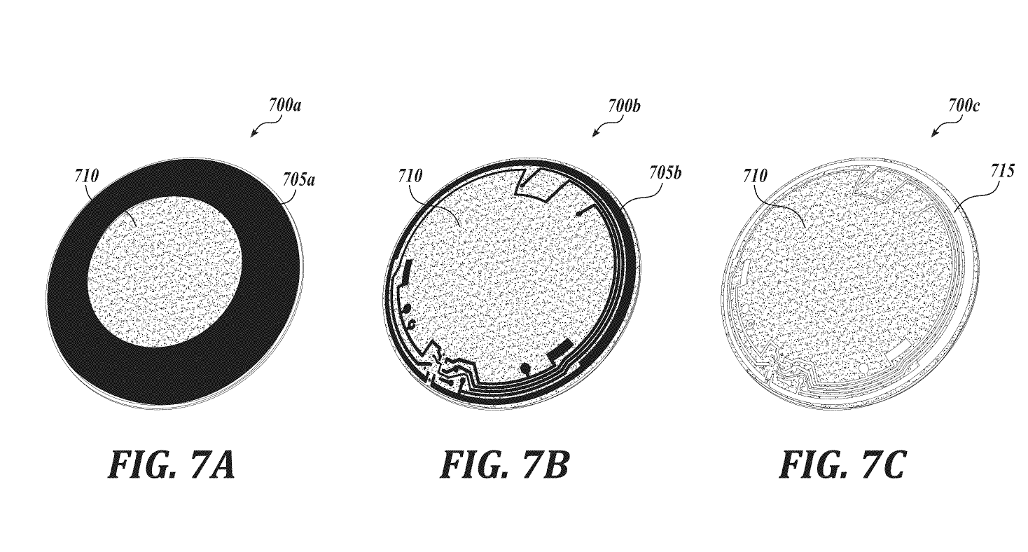

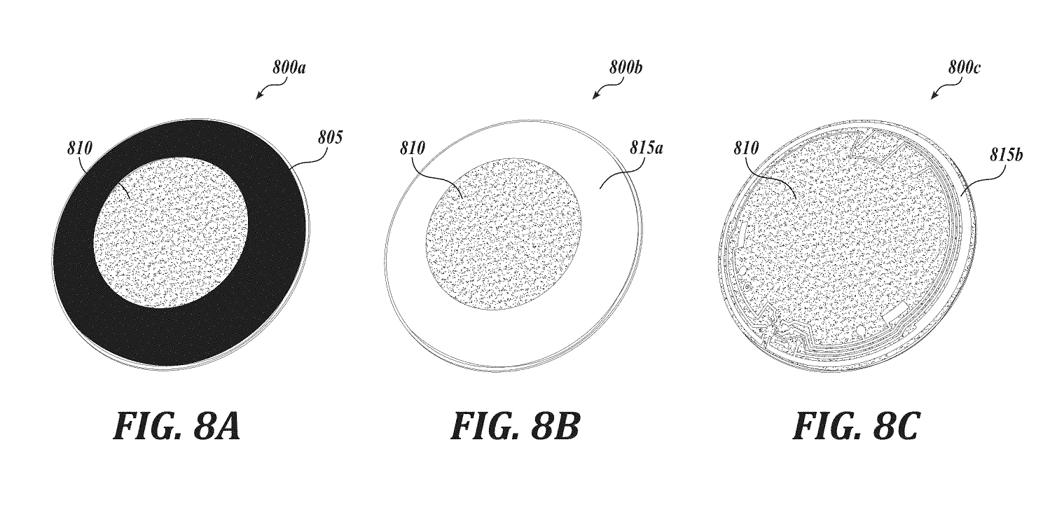

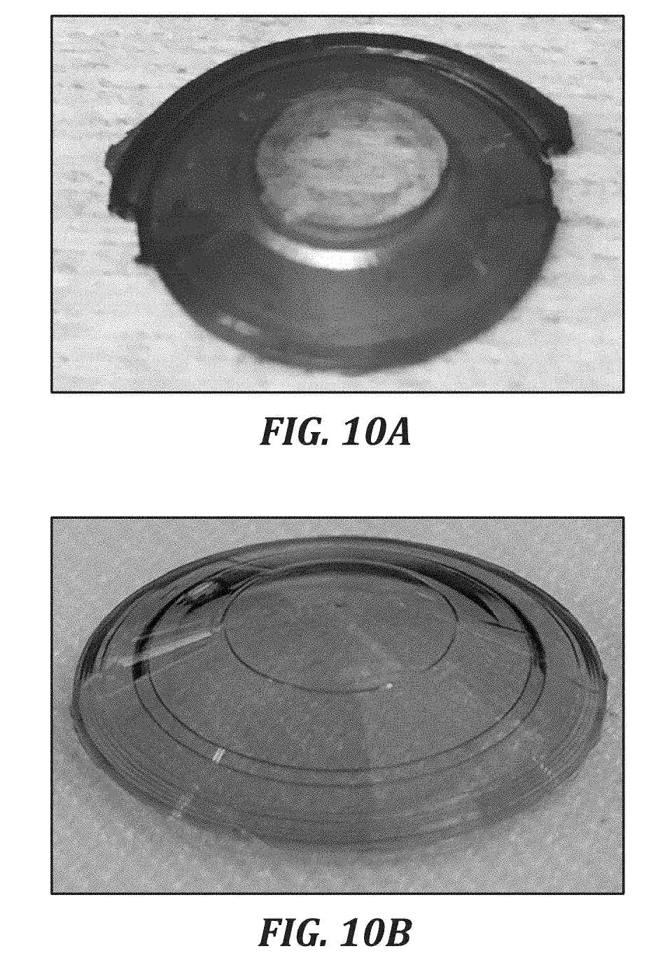

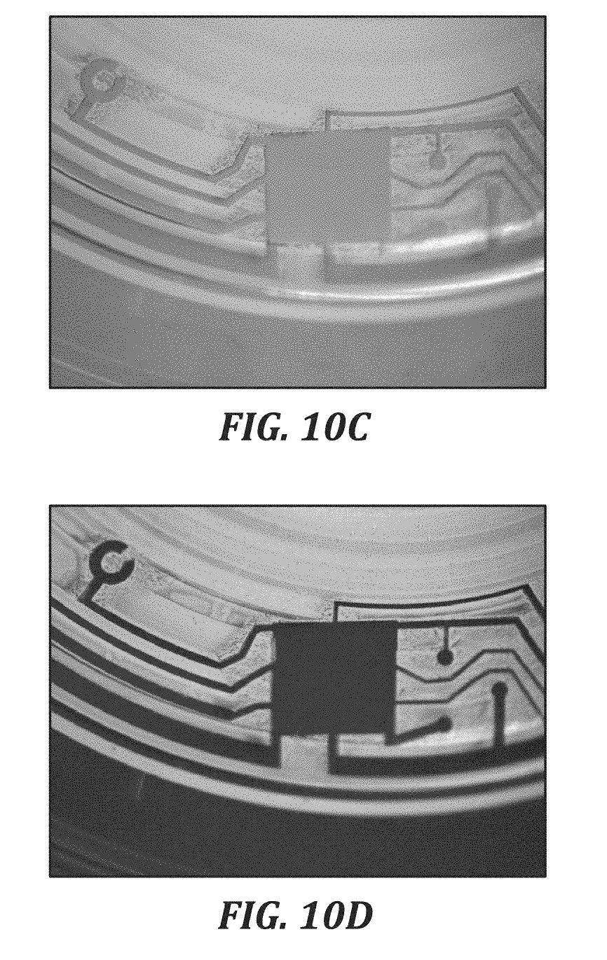

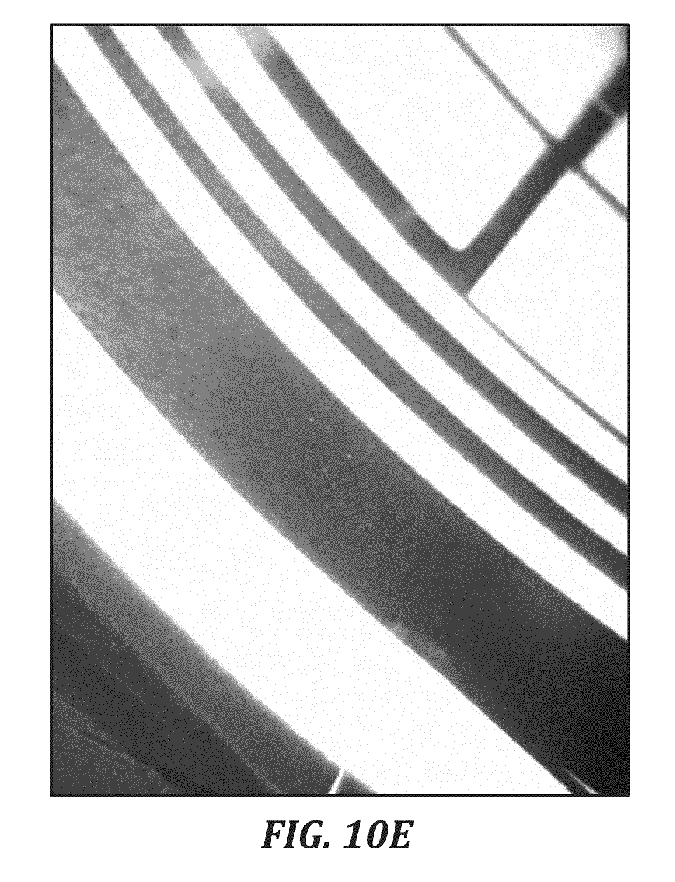

In an embodiment, forming a polydopamine layer on one or more surfaces of the ocular lens comprises contacting a portion of the ocular lens with a solution comprising polydopamine. In an embodiment, contacting a portion of the ocular lens with a polydopamine solution comprises dip-coating at least a portion the ocular lens in the polydopamine solution. In this regard and in an embodiment, a polydopamine layer defines an annular ring around an exterior portion of the ocular lens as discussed further herein with respect to FIGS. 7A and 8A. FIGS. 10A and 10B include images of ocular lenses contacted with polydopamine solutions (2 mg/mL in 10 mM tris buffer with a pH of 8.5). The ocular lens shown in FIG. 10A was partially immersed in the polydopamine solution to provide a central optic zone that is free of the polydopamine layer; whereas the ocular lens of FIG. 10B was fully submerged in the polydopamine layers.

In an embodiment, the polydopamine annular ring has a width of between about 1 mm and 5 mm. In an embodiment, the ocular lens is contacted with the polydopamine solution for a contact time in a range of 30 minutes to 24 hours. Longer contact times generally result in a thicker polydopamine layer.

In an embodiment, forming a polydopamine layer on one or more surfaces of the ocular lens comprises spraying a polydopamine solution onto the one or more surfaces of the ocular lens. In an embodiment, a mask is used to prevent a portion of the sprayed polydopamine solution from contacting a portion of the ocular lens, thereby providing a patterned polydopamine layer on the one or more surfaces of the ocular lens. In an embodiment, the patterned polydopamine layer defines an annular ring having a width of between about 1 mm and about 5 mm.

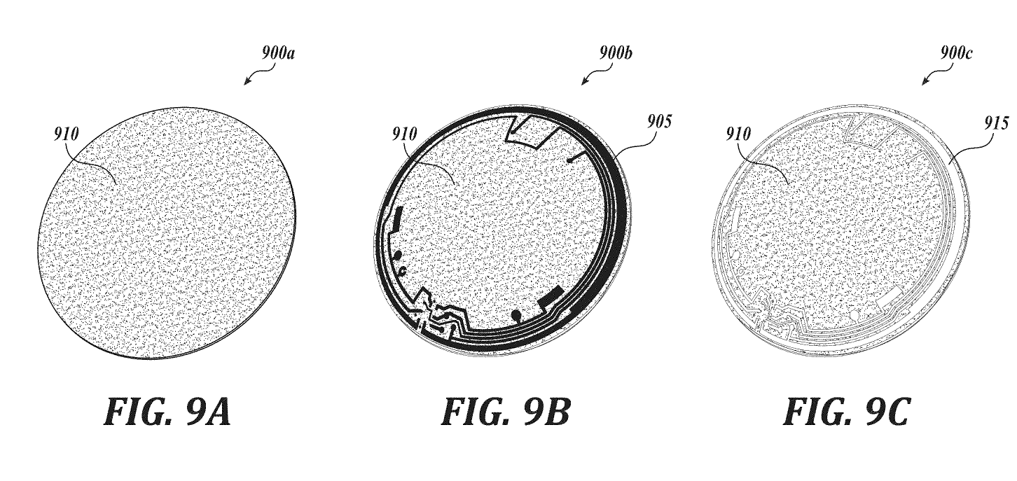

In an embodiment, forming a polydopamine layer on one or more surfaces of the ocular lens comprises contacting the one or more surfaces of the ocular lens with a stamp coated with polydopamine, as discussed further herein with respect to FIG. 9B. In this regard and in an embodiment, forming the polydopamine layer provides a patterned polydopamine layer on one or more surfaces of the ocular lens.

As discussed further herein, in an embodiment, the anterior-facing and posterior-facing surfaces of the ocular lens are generally curved and the ocular lens includes one or more locally flat regions. The polydopamine, such as from a polydopamine solution or a stamp including polydopamine, conformably couples to the generally curved surfaces of the ocular lens, as well as to any flat surfaces on the ocular lens. In an embodiment, forming a polydopamine layer includes forming a polydopamine layer on the one or more locally flat regions in addition to at least a portion of the generally curved surface, as discussed further herein with respect to FIGS. 4 and 5.

Process block 205 may be followed by process block 210, which includes patterning the polydopamine layer. In an embodiment, the polydopamine layer is patterned, for example by laser ablation, before contacting the polydopamine layer with a metal precursor solution. In an embodiment, process block 210 is optional. FIG. 10C includes an image of a polydopamine layer disposed on a surface of an ocular lens that has been patterned through laser ablation. As shown, the patterned polydopamine layer includes features in the general shape of metal traces to be deposited on the patterned polydopamine layer.

Process blocks 205 and 210 may be followed by process block 215, which includes contacting the polydopamine layer with a metal precursor solution to form a metal layer. Without wishing to be bound by theory, it is believed that the catechol-containing polydopamine layer reduces the metal ions in a substrate-catalyzed electroless deposition process, thereby forming metallic traces directly on the polydopamine layer. In an embodiment, contacting the polydopamine layer with a metal precursor solution includes contacting a portion of the polydopamine layer on a generally curved surface. Because the polydopamine layer conformably couples with the surfaces of the ocular lens, including any curved or otherwise non-planar surfaces of the ocular lens, the metal layers formed thereon also conformably coupled to any curved or otherwise non-planar surfaces of the ocular lens. Such coupling generally does not induce stress in the metal layers, typically resulting in fewer mechanical and/or electrical failures of the metal layers than those formed by conventional methods.

In an embodiment, contacting the polydopamine layer with a metal precursor solution also includes contacting a portion of the polydopamine layer on a locally flat surface, such as a mesa or recess, as discussed further herein with respect to FIGS. 4 and 5. In an embodiment, contacting the polydopamine layer with a metal precursor solution includes contacting a polydopamine layer disposed on a surface of the ocular lens that defines an aperture in the ocular lens, as discussed further herein with respect to FIG. 6 to provide a conductive via.

In an embodiment, an anterior-facing surface and a posterior-facing surface of the ocular lens are contacted with the metal precursor solution simultaneously, such as by dip-coating the ocular lens. In this regard, a metal layer can be formed simultaneously on, for example, the anterior-facing and posterior-facing surfaces of the ocular lens.

In an embodiment, the metal precursor solution includes metal and other components suitable to form a metal layer on a polydopamine layer. In an embodiment the metal precursor solution includes a metal salt. In an embodiment, the metal salt is chosen from a salt of nickel, copper, silver, gold, chromium, palladium, and combinations thereof. In an embodiment, the metal salt is chosen from a metal phosphate, a metal phosphonate, and a metal salt of an organic acid. In an embodiment, the gold salt is chosen from KAuCl₄, Na₃Au(SO₃)₂, and KAu(CN)₂.

In an embodiment, the metal precursor solution is an aqueous silver nitrate solution. In an embodiment, the aqueous silver nitrate has a silver nitrate concentration in a range from about 0.1 M to about 1.0M. In an embodiment, the polydopamine layer is contacted with the aqueous silver nitrate solution for a time in a range of about 30 minutes to about 24 hours. Longer contact between the metal precursor solution and the polydopamine layer generally results in a thicker metal layer.

Metal layers formed according to the methods of the present disclosure typically have thicknesses in a range of about 0.1 nm and about 20 μm. In an embodiment, the metal layer has a thickness in a range of about 1 nm to about 50 nm and is formed by contacting a polydopamine layer with a metal precursor solution. Such relatively thin metal layers are suitable to provide, for example, surface passivation (e.g. with a noble metal to prevent oxidation), a moisture barrier protection, a light and/or electromagnetic barrier (e.g. infrared barrier for silicon-based electronics), an optically reflective surface, electrostatic discharge protection, and electrical interconnects with low- to medium-resistance performance requirements (i.e. non-RF, short-distance connections).

In an embodiment, the metal precursor solution comprises a reducing agent to reduce the metal salt onto the polydopamine layer. Without wishing to be bound by theory, it is believed that in embodiments where the metal precursor solution includes a reducing agent that polydopamine layer initially contributes to substrate-catalyzed deposition, which is then continued by an autocatalytic deposition process aided by the reducing agent. In an embodiment, the reducing agent is chosen from an alkali metal hydride, an amine borane, a hydrazine, a hypophosphite, a borohydride, formaldehyde, iodide, thiosulfate, thiocyanate, and thiomalate.

Metal layers deposited from a solution including a metal salt and a reducing agent or other solution-based catalyst can have a thickness in a range of about 100 nm to 1 μm. Such moderate-thickness metal layers may be suitable for, for example, high-performance electrical interconnections (e.g. very-low- and stable-resistance paths, radiofrequency communication, and the like), high integration flip-chip bonding connection for use with chips (ASICs etc.), thermal heat sinking, and mechanical coupling to macroscale connectors (e.g. clip or zero-insertion force connections).

In an embodiment, contacting the polydopamine layer with the metal precursor solution does not include applying an external electrical bias to the ocular lens and metal precursor solution, such as by electroless plating of the metal precursor solution. As used herein, “electroless plating” refers to forming a metal layer, such as an electrode, on a substrate without application of an external electrical bias, such as to a surface to be plated and a metal salt solution into which the surface is disposed.

Process block 215 may be followed by process block 220, which includes patterning the metal layer. In certain embodiments, it may be desirable to have a patterned metal layer, such as for a microelectronic assembly including an antenna and electrodes. Based on the desirable characteristics of a patterned metal layer, in an embodiment, the metal layer is patterned by, for example, laser ablation to provide a patterned metal layer. In an embodiment, process block 220 is optional.

Process blocks 215 and 220 may be followed by process block 225, which includes electroplating a second metal layer on the metal layer. In an embodiment, process block 225 includes contacting the metal layer with a metal precursor solution and applying an electrical bias to the metal precursor solution and the metal layer to deposit a second metal layer onto the metal layer. In an embodiment, process block 225 is optional. In an embodiment, such second metal layers made by electroplating have a thickness in a range of about 1 μm to about 20 μm. In an embodiment, such relatively thick second metal layers are suitable for ultra-high-performance electrical interconnections (e.g. very-low- and stable-resistance paths, RF communication, sensors), high-current applications (milliamps for high-power applications, battery charging, fast discharge, etc.), high-integration flip-chip bonding connection for use with chips (ASICs, etc.), high-mechanical robustness, thermal heat sinking, mechanical coupling to macroscale connectors (e.g. clip or zero-insertion force connections), and the like.

In an embodiment, method 200 does not include lithographic patterning steps, such as use of photo-patternable resists, gray-scale lithography, UV exposures, planar micromachining, and the like.

FIGS. 7A-7C schematically illustrate an example method of forming a metal layer on a surface of an ocular lens, in accordance with an embodiment of the disclosure. In an embodiment, FIGS. 7A-7C schematically illustrate an embodiment of method 200. FIG. 7A is a perspective view of an ocular lens 700a including surface 710 and a polydopamine layer 705a carried by surface 710. In an embodiment, polydopamine layer 705a is formed according to step 205 of method 200, such as by dip-coating or spray-coating. In the illustrated embodiment, polydopamine layer 705a is disposed around a periphery of ocular lens 700a. FIG. 7B is a perspective view of ocular lens 700b including surface 710 and a patterned polydopamine layer 705b carried by surface 710. In an embodiment, polydopamine layer 705a is patterned according to process block 210 of method 200, such as by laser ablation, to provide patterned polydopamine layer 705b. In an embodiment, the patterned polydopamine layer 705b has features with line widths in a range of 50 μm to 300 μm. In an embodiment, the features of the patterned polydopamine layer 705b have a spacing resolution in a range of about 10 μm to about 20 μm. FIG. 7C is a perspective view of an ocular lens 700c including surface 710 and a patterned metal layer 715. In an embodiment, patterned metal layer 715 is formed by contacting patterned polydopamine layer 705b with a metal precursor solution, according process block 215 of method 200.

FIGS. 8A-8C schematically illustrate an example method of forming a metal layer on a surface of an ocular lens, in accordance with an embodiment of the disclosure. In an embodiment, FIGS. 8A-8C schematically illustrate method 200. FIG. 8A is a perspective view of an ocular lens 800a including surface 810 and a polydopamine layer 805 carried by surface 810. In an embodiment, polydopamine layer 805 is formed according to step 205 of method 200, such as by dip-coating or spray-coating. In the illustrated embodiment, polydopamine layer 805 is disposed around a periphery of ocular lens 800a. FIG. 8B is a perspective view of an ocular lens 800b including a metal layer 815a carried by polydopamine layer 805. In an embodiment, metal layer 815a is formed according to process block 215 of method 200, including contacting polydopamine layer 805 with a metal precursor solution. FIG. 8C is a perspective view of an ocular lens 800c including surface 810 and patterned metal layer 815b. In an embodiment, patterned metal layer 815b is formed by process block 220 of method 200, including patterning metal layer 815a carried by polydopamine layer 805, such as by laser ablation. In an embodiment, the patterned metal layer 815b has features with line widths in a range of 50 μm to 300 μm. In an embodiment, the features of the patterned metal layer 815B have a spacing resolution in a range of about 10 μm to about 20 μm.

FIGS. 9A-9C schematically illustrate an example method of forming a metal layer on a surface of an ocular lens, in accordance with an embodiment of the disclosure. In an embodiment, FIGS. 9A-9C schematically illustrate method 200. FIG. 9A is a perspective view of an ocular lens 900a including surface 910. FIG. 9B is a perspective view of an ocular lens 900b including surface 910 and patterned polydopamine layer 905 carried by surface 910. In an embodiment, patterned polydopamine layer 905 is formed according to process block 205 of method 200, such as by contacting surface 910 with a patterned stamp (not shown) coated with polydopamine. In an embodiment, the patterned polydopamine layer 905 has features with line widths in a range of 50 μm to 300 μm. In an embodiment, the features of the patterned polydopamine layer 905 have a spacing resolution in a range of about 10 μm to about 20 μm. FIG. 9C is a perspective view of an ocular lens 900c including surface 910 and patterned metal layer 915 carried by polydopamine layer 905. In an embodiment, patterned metal layer 915 is formed according to process block 215 of method 200, including contacting patterned polydopamine layer 905 with a metal precursor solution.

The processes explained above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, the processes may be embodied within hardware, such as an application specific integrated circuit (“ASIC”) or otherwise.

A tangible machine-readable storage medium includes any mechanism that provides (i.e., stores) information in a non-transitory form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. An ophthalmic device shaped for mounting in or on an eye, the ophthalmic device comprising:
an ocular lens;
a polydopamine layer disposed on a portion of one or more surfaces of the ocular lens; and
a metal layer disposed on the polydopamine layer.

2. The ophthalmic device of claim 1, wherein the ophthalmic device is an accommodating contact lens further comprising:
a controller disposed at a periphery of the ophthalmic device; and
an accommodation actuator disposed in a center region of the ophthalmic device and in conductive communication with the controller through the metal layer.

3. The ophthalmic device of claim 1, wherein the polydopamine layer is disposed on a generally curved surface of the ocular lens.

4. The ophthalmic device of claim 3, wherein polydopamine layer is disposed on a locally flat portion of the ocular lens.

5. The ophthalmic device of claim 4, wherein the ophthalmic device further comprises an electronic component including a generally flat surface coupled to a portion of the metal layer disposed on the polydopamine layer disposed on the locally flat portion.

6. The ophthalmic device of claim 3, wherein the locally flat portion is recessed from the generally curved surface.

7. The ophthalmic device of claim 3, wherein the locally flat portion extends above the generally curved surface.

8. The ophthalmic device of claim 1, wherein the one or more surfaces includes a surface that defines an aperture in the ocular lens, and wherein the polydopamine layer includes aperture polydopamine disposed on the surface that defines the aperture.

9. The ophthalmic device of claim 8, wherein the metal layer includes a conductive via disposed on the aperture polydopamine, and wherein the conductive via is in conductive communication with an electronic component disposed on a first side of the ocular lens and with an electronic component disposed on a second side of the ocular lens.

10. The ophthalmic device of claim 1, wherein the metal layer is disposed on a peripheral portion of the ocular lens to provide an optically transmissive center of the ocular lens.

11. A method of forming a metal layer on a surface of an ocular lens comprising:
forming a polydopamine layer on one or more surfaces of the ocular lens; and
contacting the polydopamine layer with a metal precursor solution to form the metal layer.

12. The method of claim 11, further comprising patterning the polydopamine layer.

13. The method of claim 11, further comprising patterning the metal layer.

14. The method of claim 11, wherein the metal precursor solution comprises a metal salt and a reducing agent to reduce the metal salt onto the polydopamine layer.

15. The method of claim 11, wherein contacting the polydopamine layer with a metal precursor solution does not include applying an external bias to the ocular lens and metal precursor solution.

16. The method of claim 11, further comprises contacting the metal layer with a metal precursor solution and applying an electrical bias to the metal precursor solution and the metal layer to deposit a second metal layer onto the metal layer.

17. The method of claim 11, wherein the surface of the ocular lens is generally curved and includes one or more locally-flat regions, and wherein forming a polydopamine layer includes forming a polydopamine layer on the one or more locally-flat regions.

18. The method of claim 11, wherein contacting the portion of the ocular lens with a polydopamine solution comprises dip-coating at least a portion the ocular lens in the polydopamine solution.

19. The method of claim 11, wherein forming a polydopamine layer on one or more surfaces of the ocular lens comprises spraying a polydopamine solution onto the one or more surfaces of the ocular lens.

20. The method of claim 11, wherein forming a polydopamine layer on one or more surfaces of the ocular lens comprises contacting the one or more surfaces of the ocular lens with a stamp coated with polydopamine.
